SEQUENCE LISTING (54 sequences)

SEQ ID NO: 1 - DNA, 30 bp, Homo sapiens
gtggatgttgcagtgagccaagatcaagcc

SEQ ID NO: 2 - DNA, 33 bp, Homo sapiens
cgtcctaagtaaacccaggatatgtgtaatgcc

SEQ ID NO: 3 - DNA, 24 bp, Homo sapiens
ctccagcctgggccacagagtgag

SEQ ID NO: 4 - DNA, 51 bp, Homo sapiens
tgtaaaacgacggccagtcgtcctaagtaaacccaggatatgtgtaatgcc

SEQ ID NO: 5 - DNA, 40 bp, Homo sapiens
accaggaaacagctatgaccctatcgcctccgcctagcag

SEQ ID NO: 6 - DNA, 38 bp, Homo sapiens
actgtaaaacgacggccagtctcccacccatctcgccc

SEQ ID NO: 7 - DNA, 24 bp, Homo sapiens
attggacttgtagtgtgcttacat

SEQ ID NO: 8 - DNA, 24 bp, Homo sapiens
tccaaatacatgatttgaatgaac

SEQ ID NO: 9 - DNA, 19 bp, Homo sapiens
catgatttgaatgaacagg

SEQ ID NO: 10 - DNA, 30 bp, Homo sapiens; identical to SEQ ID NO: 1

SEQ ID NO: 11 - DNA, 33 bp, Homo sapiens; identical to SEQ ID NO: 2

SEQ ID NO: 12 - DNA, 24 bp, Homo sapiens
ctccagcctgggccacagagtgag

SEQ ID NO: 13 - DNA, 51 bp, Homo sapiens; identical to SEQ ID NO: 4

SEQ ID NO: 14 - DNA, 25 bp, Homo sapiens
tgctaggtgttcaaggagcatgcaa

SEQ ID NO: 15 - DNA, 25 bp, Homo sapiens
ttcaggctgtcttcttggtgcaagc

SEQ ID NO: 16 - DNA, 25 bp, Homo sapiens
gggaccgtaatggctggggaattgt

SEQ ID NO: 17 - DNA, 15 bp, Homo sapiens
gatctggaggtgagg

SEQ ID NO: 18 - DNA, 635 bp, Homo sapiens
  UNSURE 16: any
tttttttctgttgcancaatgtaggaaggtttattaaccatccttttgctagtgacattatgccatatgttctatggaatgaaaaagtacaagaggccctgcccttgagatcttatcaactaacatgatttatagcaggncctcaataagtggattcttgggtgtttaccttttgtgtaatcagaatgtagatgatgaagaagatacttaacatgcattttatatctaggtaattagaaaatgtgaatagctgtttctcacttgtgttttctgcttgattgctcttctacttgcaaggcttaggtaataaggtcgagatacttatctggtttgatcttaaatgtttgaattcatataatttttaagaaatggctgctttaaagttggttgccagtaagtaataaaggatttattgtttgagtgaagaagaaataacatagttctcttaattttataattattttccagaattataaggaacagtatcaaatagtcatatgtatgggacactgtgcatacaaagcagggtttatagcacacttttccttaaaatcttttcctaaaaatacaatgagctgtatactaagtgttcacccttgatattccttccaggatccaaggattctgtagctaca

SEQ ID NO: 19 - DNA, 506 bp, Homo sapiens
gttagtgaagttaatttgatactgactaaagtaaattacattttcaatttttgaagagcccttaagcccctatagggagcacataatttttaaaagttagagtaaaatatttattttagtattttggaacttacctcaaatttctgcttacatggaatgcactggaaatgctcttattttgctttgtctttacaaatgaatgtaattgactttatttgagaaatacatcttttataagtgactaatagtcaaaaatgattgtgggccgggtatagtgactcatgcctgtaatcccagcactttgggaggccaaagcaggagaattgtttgagcctaggagttcaagaccagcctgggcaacctggacaacatagtaagacccaagtctttaaaaaaaaattaaaggccgggcaccggtggctcatgtctgtaatcccagcactttgggagggcaaggcgggtggatcacgaagtcgggagaatcgagaccattctggctaacatgg

SEQ ID NO: 20 - DNA, 230 bp, Homo sapiens
  UNSURE 26: any
aaaaaaaaaaaattgtggtgatgtantggcttnccacagtggtttgattaaaagttggatttaatttttgatttgtaggtttgatatttttattggcttgtagtgtgcttacatttatgttctcatgactatataaatgaattacacatgcaaaataaaaattcttagttttgattacttattttaaaagtcaaagctaatggaatttccttttctttctctcctattag

SEQ ID NO: 21 - DNA, 280 bp, Homo sapiens
ctaaaatgacaaaagttcaatactaaaaaaactttatcctttactacacaaataataacagtgtcacacagcattctgagataatactagtttactccaaattattaaggtctcaaatttcagaatgtctaattgccaataataaggaaactattcaatgcatgcagcacactttcagcaatacacatatttccaaatacatgatttgaatgaacaggtttatttccacagcaaatcaaaaatctgatgacaccagaagttaaatatgtaaaactattac

SEQ ID NO: 22 - DNA, 1222 bp, Homo sapiens
  UNSURE 604: any
gtaagtatttaaaacctaaatatactttctgtcaaaatacattttaaaaaacttttcttccccatgctgtaaaggtacattttcaaaagttaagaaaataaggggaaatttttttgtataattttactattagctaattttaataactattaacattttggcatatatccttttctactgtttttatacttaaagaaaatatctgatatcatatatattgttttataatttctttatgcttaataatagtttatcaacatctttccatgtcccttttttttttttttgagatggagtttcgctcttgtcacccaggctggagtgtaatggcacgatcctggctcactgcaacctccacttcccgggttcaagcagttctcctgcctcagcctcctgagtagctgggattcaggcacctgccaccatgcccatttaatttgtgtatttttggtagagactgtgtttcgcatgttggcaggctggggcaaactcctgcctcaagtgatccgcctgccttggcctccaaagtctgggattataggcgtgagccctggcccggcctcatgtccttaatgtaaactaaatggttggaatggctatatgnatctcttctgctaaacgcttggagntattaatcatctaatgtggactgttaggtatgtaattttttttattaataccaccactgtgatgaatgtctttgaacgaaatttttgttcatatttgtaatcattttcttaagatacattcctagaagtgagacagtggttttgctttttttagagctttgcttttttttttttaagagctttttagtgctactattgccaatttagtttacagaaagtttgtttagtttatccttccgcaggtagtgatcaatgaaaatttttatatattccactttttttccctaatggtaatccaaggagatattttttactaaggatgatactttgatacaaaattatcaaaaggtgtttaaatgtaaatatacttacattttaacattaaaaatatttttaacaaatattttgagcaactactatgtttagctttgaggatgccaaagaaatatagggtatacttttttgtctgcagaaaggtacacatactacaggatcatacagtatggagggggaaaggttttgttctaaaaagaatttttttaaaatcatactttttcccgttaaatttatctgatcattttgttcttttccag

SEQ ID NO: 23 - DNA, 849 bp, Homo sapiens
  UNSURE 1: any
nnannnttattatccgatggantgngaatngnnttcctttnttcagncnacattaataggaaggattaatngcgtttcttcatagcacaagatttaagaattgcccaaagttttaagtntaattctcaagcccaagactggtctccataagtgccccaggaacagtcccctgtatctaacaactcaattatgattctgtagctactggaatttggaattncccccatttttctttttgaaagttttcagaacttntggtnataataatttttgggtnaataagagtattttcctagtgaaacacatcagagagcagaacaagatctaatggaagagaaacccagggtagattgattgattgattgatttgagatgtcgtctcacnctnctacccngngctgnnttgctcnnggtnagatcttnggtcacccaccncccttntcactcntggctncgcacgggntccttctgccntcncccacctnnncagcnnncgattccnccntggtncccctnccntctcgcnnctnaatcttntcctgttcncatgctctncncnntattctttngccantgnttggctcaagactcgccccttcatnttctttgcaccntcaanncgnacccncccgcnctcccccttccctagtgcgcgngnatgttcngcttnganccnctnctncctgngccatntttnntcttanaccccnnaaatncacncttggccctttntnntcctattnttacncttcnntctttnacgtgncactntntancttttnacnnaccctnancctctttnccacntntcctnttnnctatnnctctgcctcttnanntnccccacct

SEQ ID NO: 24 - DNA, 730 bp, Homo sapiens
  UNSURE 8: any
cgaaaaangccccattcncccnatattggnaagaagggggatcnttgacatatacaaggaataggtgggtttatgaatcnaatangtctcataaatttcnaactaagcttctgtcaggtaggtaaaatatagaangtgnaggatttaattaggggattaaatgccagagtaatnccctncctcaaaggaatantcctaccaaatatcnnattcagggaaaggaatcagggcnctatatgttcttttttaaaattgggcngggcccagggctcncacctgaattccagcacttcggnagnccgaggtgggcagatcncctgaggcaggagctccagaccagcctggccaacctggtgaaacccagtctctactaaaaatacaaaaattagctgggcatggtggcgggtgcctgtaatcccagctactcgggaggctgaggcaggagaattgcttgaacccaggaggcagaggtngcagtgaaccaagatcacaccattgcacattgcactccagcctgggaaacaaagtgagactacatctcaaaaaaaaatttttttaaatcctttatattacaatcatactttgtatcttgaatacctgttagttttatcatattgtatattttactctttgaatagtaatttgatattaatataagccataggatgctctaacatttaaaaaagttgttctgtcccttgccttcattgatatgtttgatctgttttag

SEQ ID NO: 25 - DNA, 491 bp, Homo sapiens
  UNSURE 88: any
gtttgtaagcaatttttgccatattttaaaataggtatgtcgctaaagaggaaaaagaacatcttggatttgtattatttattgttanattctgacttttaaattactcttaaaattttttattatattagtgtgtgggtatatctggcctgtttgctttggtggaaacttagcagcaggttactgatttatttttcactcctgccanctactttgtgngcatnactntgtgatatttttattcattngatntctttngttnggttttttanccaacattgcattccaaggttggtctggaaaacactttccagcccctgctgctacttaagactcaattggngacttgggtctttgtgtnttaattntcatgtagggacgaaaagagtcaggaattgggaccagatctggagtaaggattcacctnataaggnggattgggaantatttaaatccggataagtaagccggaccatcttcagcaagtaccc

SEQ ID NO: 26 - DNA, 715 bp, Homo sapiens
cgttttctattttgagatcccccaaatgaatccctttaacaagtgtgaagtacaatatgtttggttaatttttagtcttaagttgggcattttgtattatatcattttccaacagatcaatgaaataaccaaaattataagaacttcagtaggcatcatagaggttatattgaaaattagagtttgttggtacacaaaaaatattattttgaccttatatcaggactggcataactggcaggatattctacttatataaaaaatccttggttaattggcaaattgcttttctcctaacaagtgggattagatcaatagtgtcactggggttttgctctgttagagtagcctgtcctctccttatttaataataaggcactactgcttgacaaaaaggaattggaaacacatatgttttatcaattgtgtattaaacactaccattctgcctggcattgtgatatggggacatgagagaaggcaagagctttgctcttgagggacttagagttctgttgttattcctgctgtttcctaaggcttggcatcacctctaagttgctaattctatttccagtaagtggcaaggagcttaatgtactaatattttcatgttttgtccacctgcaggaagacaaatatatccttgtgatatatgcagcataaaaaataacgtagactttactagttgtatctttaatttttctctaaccag

SEQ ID NO: 27 - DNA, 109 bp, Homo sapiens
gtaagcccaattcttacatatggcactagtagaagagtaagatttctgcttacacagtttacctaaacagaatcaataccttctaatgtcacactgacttaatttgtag

SEQ ID NO: 28 - DNA, 414 bp, Homo sapiens
gtaatgacatggttttcttcttcttttagtatcctcagttccaatctcattatttttaagatttctttttttctacaattttggcccattcaatgatattgcacccccttcttcctttttttcttaatgtgttcatttctttgaggctcctggtctttattagcccctctctcctaaacagactttttaagttcccccaccttatctctcgttgaaagcctgttctttggggtgttttcagtagttcagtggggtcactactttagttagttgcatagcaagcttggggctttttttttttcatggtaaggggaagctatgagagataatgtctggctgtccagttgctagggataagaaatttaagttctattgatatgcagaggatacattactttaaaaattttatttcag

SEQ ID NO: 29 - DNA, 118 bp, Homo sapiens
gtaagttaattgaaatctactttgtgatatattaatcataacactctatgctaatgtaagtttagattgtgtcctttacatttctgaataagattttaatttgctttcttttatttag

SEQ ID NO: 30 - DNA, 108 bp, Homo sapiens
gtaacttgttattctcttcgctttcaatccttcattgctttgtcaaaaagtagtctgttttcaaaattatgtgccgtgttgtgagatttcttttgatttcttgaacag

SEQ ID NO: 31 - DNA, 728 bp, Homo sapiens
  UNSURE 49: any
gtgaggatgataacataaactccaatgtggcatttttcattacaaaggngcttngnnaaggangaaaaatctagtatctgctgaacactncagctaagttctgggcacggtgtancatgactaacagatactatcttctttctttatttcacacaaccttgagaggtaggtncaattatctatttttcagatgagaacattgaggctccaatatgtttaatttcccaaagnagtccctctnggaaatgatnaagctgatagnagggtccaagattttctgactccagagtcaaaactctttctagtttattactgcttatcatagagatgagtgactactgtatnctcataggngtgntgaggcctagaaagagtttaccacagagacaagtttcaaagatagangaaagtttgttttngtnttgtttngnngctggatacccatgaggaagtttgcttttctttctgacatttgaacaggaccttntgcctacatgaccatatgaatctacttatgctttcatgcaaanaatcatggttccatncatgtctgcttnacacgggtgtttcttttaanncacaggntaattncgtttaattgggnaaaatgccattttttggccagccttttttgagggtttcttggccaaanttttttttgnatantnntgatnnataatgattattatcnctngntttggagacaaaanntnncttttttcccccag

SEQ ID NO: 32 - DNA, 30 bp, Homo sapiens
caatttcattttttttctccatttctttag

SEQ ID NO: 33 - DNA, 358 bp, Homo sapiens
gtatgttattttctcgattaagagagatttgctttgtatgtttttaatcttttttcttgattagtttcatatatgtacatagttttataaaacattttccttttaaatcattttatcctaattttttattctgcttatgatgtaggtcatagaaattaaaaatatatttcctgcttttatagtcattactcaaagattttagtattttaaacactttttaaaggtgaattaaacattttgtttaaaaagaatacatactaaaggattaagtttgaagatagttatactgacaagctgagataaaattttgtgcatttactatatagattttcatttggtgcctgactttaccttttag

SEQ ID NO: 34 - DNA, 150 bp, Homo sapiens
gtaagttggcatttatatatttgccagtttaaaaatacatcataagtaaggcaatgagaagagttttaaggacaattagtgataccttttgggtcaagcatgagcatttttgggtaacatgtgcttgcttctctaacatatactgtgtag

SEQ ID NO: 35 - DNA, 246 bp, Homo sapiens
  UNSURE 106: any
gtgtgtgagtggatttgtatgtacagttatatctatttgtttattttagaaccagtgtcattttctgtgattaccaaacataattgttaacatattacctgctaangagcacataacagaatatcaactttaaagccattcattnaaaatgagtaatatttatgctgggnnggggggaaaaaaagaatgtngatncaaatgaatngctccncagaggtaaattagtaagaaaaaaaaaaagggggg

SEQ ID NO: 36 - DNA, 594 bp, Homo sapiens
  UNSURE 22: any
aaaaaaaaaaaaaaagtgtaanctagtaatttttgattagatgttactttgcctagganagaactgttttagaaaaaaagatttttcaaataggagagaaatattagtataataagacttccttcaaataaagaaaattaataaagtagcataatcaacncaaatgatanccatagtatagttcaagcttaacacatttgtttttatgtgaactgtgtaagtttattaagaaataattgtgactgggcgcagtggctcacgcctgtaatcccaacactttggggaggccaacgcgggcagatcacttgaggccaggagttcgagaccagcctggccancatggcgaaaccctgtctctactcaaaatacaaaaattggctgggcatggtggcccgcgcctgtaatcccagctactcgggaggctgaggctggagaatttcttgaacccgggaggtggatgttgcagtgagccaagatcaagccactgcactccagcctgggccacagagtgagactccgtctcaaaaaanaacaaaaaacaaagaaataataataataaaagaataaaacacagtctttgcatctttnttatag

SEQ ID NO: 37 - DNA, 357 bp, Homo sapiens
  UNSURE 6: any
gtaagnntngccagantntntnaangtccttttattaantntttnnncttttataaaaaacaaatcagcccttttgttgatggncattcnttncnttnganngattcantttanantnggcnttacacatatcctgggtttacttaggacgggnaacantnttagtntngacatttcaaaactttntccagtcaananaccncntttgaggctgacctctncaagattngtntttaanancnccantatnttttcngccttnggnaggccnnggcaagaagnttgnttggggtntgaagntnaanaccagccngggcnacacananagatgctntntctanaaacaataaaaaaaaa

SEQ ID NO: 38 - DNA, 342 bp, Homo sapiens
  UNSURE 39: any
gtgagtgactggatggataatttatcttttttattttgnaatctttaattgtatttaaaaatgggggaaaggagtattaacattttaaataaagttaaatatatgggacagtgttttccatcaaagatgactgttgtaccttgcccatctgtctgtgtgnatcatccataggaacaaactttactgattttttttaattttttttattttttaatggaggacagggcttaaatggggccacatctaaactttgttttctggaggttcagaaagatagatttgggtaacattcccctgaaccttctggaggaacatctaaatgtacacagctctgttttgtag

SEQ ID NO: 39 - DNA, 87 bp, Homo sapiens
gtgagctctccagcctccacttctcttgtgttacgtctttctaagtgaaagaagtatatggtatattttttcttttcttgtttccag

SEQ ID NO: 40 - DNA, 428 bp, Homo sapiens
gtatgatgtatcaggcatagagtccacaagcctagttctgactctctgggtttctctttctatctgagactatgtatcactcacctctattttaattggtcttttccaaactcttttgtcatatcagcctaatccattgtgtccaaataagcatgtttaagcttatgcttagataagaaagtagatgaagagagcaaatgaatgttcatctactgagttaagggtactgccagtcaggctgtgaatattatgttagctatggtattatgcactgtcaggtgtggctgtcaagtcttggaaagttagtgcttccagtggagtctagttctattctgatgccattatagttgccctgtttttagttgatttagtaagaaattggtcatgattttaagggtgaatcttgttgtgtctctccctggctacag

SEQ ID NO: 41 - DNA, 148 bp, Homo sapiens
gtaagactcaaagatatatttaacatgttccccctatacttcaaaaaatatgcagtgtaaaaacttactattcatctactgtagttccaagttaaaattctacactcctgatatttatatattgctactttgtcattttctaccatag

SEQ ID NO: 42 - DNA, 42 bp, Homo sapiens
aacgacggccagttcaggagctccagccagctggcaacctgg

SEQ ID NO: 43 - DNA, 31 bp, Homo sapiens
ggcaggagtgaaaaataaatcagtaacctgc

SEQ ID NO: 44 - DNA, 2904 bp, Homo sapiens
ttcggtggcctctagtgagatctggaggatccaaggattctgtagctacaatgttgtcaagactttttcgaatgcatggcctctttgtggcctcccatccctgggaagtcatagtggggacagtgacactgaccatctgcatgatgtccatgaacatgtttactggtaacaataagatctgtggttggaattatgaatgtccaaagtttgaagaggatgttttgagcagtgacattataattctgacaataacacgatgcatagccatcctgtatatttacttccagttccagaatttacgtcaacttggatcaaaatatattttgggtattgctggccttttcacaattttctcaagttttgtattcagtacagttgtcattcacttcttagacaaagaattgacaggcttgaatgaagctttgccctttttcctacttttgattgacctttccagagcaagcacattagcaaagtttgccctcagttccaactcacaggatgaagtaagggaaaatattgctcgtggaatggcaattttaggtcctacgtttaccctcgatgctcttgttgaatgtcttgtgattggagttggtaccatgtcaggggtacgtcagcttgaaattatgtgctgctttggctgcatgtcagttcttgccaactacttcgtgttcatgactttcttcccagcttgtgtgtccttggtattagagctttctcgggaaagccgcgagggtcgtccaatttggcagctcagccattttgcccgagttttagaagaagaagaaaataagccgaatcctgtaactcagagggtcaagatgattatgtctctaggcttggttcttgttcatgctcacagtcgctggatagctgatccttctcctcaaaacagtacagcagatacttctaaggtttcattaggactggatgaaaatgtgtccaagagaattgaaccaagtgtttccctctggcagttttatctctctaaaatgatcagcatggatattgaacaagttattaccctaagtttagctctccttctggctgtcaagtacatcttctttgaacaaacagagacagaatctacactctcattaaaaaaccctatcacatctcctgtagtgacacaaaagaaagtcccagacaattgttgtagacgtgaacctatgctggtcagaaataaccagaaatgtgattcagtagaggaagagacagggataaaccgagaaagaaaagttgaggttataaaacccttagtggctgaaacagataccccaaacagagctacatttgtggttggtaactcctccttactcgatacttcatcagtactggtgacacaggaacctgaaattgaacttcccagggaacctcggcctaatgaagaatgtctacagatacttgggaatgcagagaaaggtgcaaaattccttagtgatgctgagatcatccagttagtcaatgctaagcatatcccagcctacaagttggaaactctgatggaaactcatgagcgtggtgtatctattcgccgacagttactttccaagaagctttcagaaccttcttctctccagtacctaccttacagggattataattactccttggtgatgggagcttgttgtgagaatgttattggatatatgcccatccctgttggagtggcaggacccctttgcttagatgaaaaagaatttcaggttccaatggcaacaacagaaggttgtcttgtggccagcaccaatagaggctgcagagcaataggtcttggtggaggtgccagcagccgagtccttgcagatgggatgactcgtggcccagttgtgcgtcttccacgtgcttgtgactctgcagaagtgaaagcctggctcgaaacatctgaagggttcgcagtgataaaggaggcatttgacagcactagcagatttgcacgtctacagaaacttcatacaagtatagctggacgcaacctttatatccgtttccagtccaggtcaggggatgccatggggatgaacatgatttcaaagggtacagagaaagcactttcaaaacttcacgagtatttccctgaaatgcagattctagccgttagtggtaactattgtactgacaagaaacctgctgctataaattggatagagggaagaggaaaatctgttgtttgtgaagctgtcattccagccaaggttgtcagagaagtattaaagactaccacagaggctatgattgaggtcaacattaacaagaatttagtgggctctgccatggctgggagcataggaggctacaacgcccatgcagcaaacattgtcaccgccatctacattgcctgtggacaggatgcagcacagaatgttggtagttcaaactgtattactttaatggaagcaagtggtcccacaaatgaagatttatatatcagctgcaccatgccatctatagagataggaacggtgggtggtgggaccaacctactacctcagcaagcctgtttgcagatgctaggtgttcaaggagcatgcaaagataatcctggggaaaatgcccggcagcttgcccgaattgtgtgtgggaccgtaatggctggggaattgtcacttatggcagcattggcagcaggacatcttgtcaaaagtcacatgattcacaacaggtcgaagatcaatttacaagacctccaaggagcttgcaccaagaagacagcctgaatagcccgacagttctgaactggaacatgggcattgggttctaaaggactaacataaaatctgtgaattaaaaaagctcaatgcattgtcttgtggaggatgaataaatgtgatcactgagacagccacttggtttttggctctttcagagaggtctcaggttctttccatgcagactcctcagatc

SEQ ID NO: 45 - DNA, 1227 bp, Homo sapiens
tggtcccctatcgcctccgcctagcagctgccatcggtgcgcccccacagctctaggaccaataggcaggccctagtgctgggactcgaacggctattggttggccgagccgtggtgagagatggtgcggtgcctgttcttggccctgcagagagctgtgggcggttgttaaggcgaccgttcgtgacgtagcgccgtcaggccgagcagcccccaggcgattggctagacaatcgaacgatcctctcttattggtcgaaggctcgtccagctccgagcgtgcgtaaggtgagggctccttccgctccgcgactgcgttaactggagccaggctgagcgtcggcgccggggttcggtggcctctagtgagatctggaggtgaggcgggcggtgaccgagaagaggggcaggggcggcggggagcggggcgagatgggtgggagcggggtttgggctgtgttggtggcaattctggagcttccctcggccctgggaagtggctaccggcagctcctgcggacctggagggggctgcggttgcgctttgtcggtgtggcagctcggacccgcggggactgcaaggaatgtccttgaggcccggcaggccgagcggcggccggcatcagtgccggagtaacccggggtcccggggtgggcttgagaggcgggcggcggtctggcctcttcgtgactgcggtcatcatcggtggacccgcggggcgtagctgcgttcatcgtccctgttcagtcagagtaggcagtgctggctgcacggtcacgaaaatcggggcggaaagggtgtcaggcagggtgacctcggaggcccctggattcgagaaatgctaggggtctatggggctgtcgggccggcagctcgcagggcagacgggagaagcgcctgcatcccgggatccggcattctcgccaggaactgctgttcgttagcacctttcttttaggtgacgggaaagatctctgtaaatactgctgactaacttagaaccatgaaagaaccgtggattggtgtagatgtgtctggttatttacaggagaacggcttgagaggatgcggagcccaacgtgggacttcgcacaatgactcaaaagattcttctccctctttttttttttttttttttggtaaggggtgtagtctccttggtgctgatattcttttaggaaaaatgtaccttggagatacaaatatagaacagttaatttctgcag

SEQ ID NO: 46 - DNA, 42 bp, Artificial Sequence
  Description of Artificial Sequence PCR primer
tgtaaaacgacggccagtaggaatactattcacattcctatc

SEQ ID NO: 47 - DNA, 44 bp, Artificial Sequence
  Description of Artificial Sequence PCR primer
tgtaaaacgacggccagtctttgggcaattcttaaatcttgtgc

SEQ ID NO: 48 - DNA, 30 bp, Artificial Sequence
  Description of Artificial Sequence PCR primer
gcaaactcctgcctcaagtgatccgcctgc

SEQ ID NO: 49 - DNA, 31 bp, Artificial Sequence
  Description of Artificial Sequence PCR primer
gttcaagcagttctcctgcctcagcctcctg

SEQ ID NO: 50 - DNA, 22 bp, Artificial Sequence
  Description of Artificial Sequence dye terminator sequencing oligo
agaaaggtacacatactacagg

SEQ ID NO: 51 - DNA, 25 bp, Artificial Sequence
  Description of Artificial Sequence PCR primer
atgttgcagtgagccaagatcaagc

SEQ ID NO: 52 - DNA, 28 bp, Artificial Sequence
  Description of Artificial Sequence PCR primer
aacggatataaaggttgcgtccagcagt

SEQ ID NO: 53 - DNA, 28 bp, Artificial Sequence
  Description of Artificial Sequence PCR primer
aacggatataaaggttgcgtccagctcc

SEQ ID NO: 54 - DNA, 291 bp, Homo sapiens
ttcacatttatttttctttttatggtgtattagtgcaagcctgtctttgtattgtaaaatctaatgatacggtatttatattatttttgtttggcattttttgcattaaatgaattattttgcagaggtatcttttaattaaaaactacagtgatttaatttaaaaattacattattttagcttagcattgtttgtattaaatggtttataacatgaaatacagtccttcaagtcttctgtttcatctctctctctgaccacaatttcattttttttctccatttctttag

(19) United States
(12) Patent Application Publication
(10) Pub. No.: US 2003/0158081 A1
(43) Pub. Date: Aug. 21, 2003

(54) Genetic polymorphisms in the human HMG-CoA reductase gene and their use in diagnosis and treatment of diseases

(75) Inventors: Ruth E. March, Macclesfield (GB); Sarah M. Thornton, Macclesfield (GB)

Correspondence Address: FISH & RICHARDSON PC, 225 FRANKLIN ST, BOSTON, MA, 02110, US

(73) Assignee: AstraZeneca UK Limited, a English corporation

(21) Appl. No.: 10/196,095

(22) Filed: Jul. 15, 2002

Related U.S. Application Data
(63) Continuation of application No. 09/597,835, filed on Jun. 19, 2000, now abandoned.

(30) Foreign Application Priority Data
Jun. 22, 1999 (GB) 9914440.4

(51) Int. Cl. A61K 31/00; C12Q 1/68; G06F 19/00; G01N 33/48; G01N 33/50; C07H 21/04

(52) U.S. Cl. 514/1; 435/6; 702/20; 536/23.2

(57) ABSTRACT

This invention relates to polymorphisms in the human HMG-CoA reductase gene and corresponding novel allelic polypeptides encoded thereby. Particular polymorphisms are described in the promoter, exon 15 and introns 2, 5, 15 and 18. The invention also relates to methods and materials for analysing allelic variation in the HMG CoA reductase gene, and to the use of HMG-CoA reductase polymorphism in the diagnosis and treatment of HMG-CoA reductase mediated diseases such as dyslipidemia and other cardiovascular diseases such as myocardial infarction and stroke.

This invention relates to polymorphisms in the human HMG-CoA reductase gene and corresponding novel allelic polypeptides encoded thereby. The invention also relates to methods and materials for analysing allelic variation in the HMG CoA reductase gene, and to the use of HMG-CoA reductase polymorphism in the diagnosis and treatment of HMG-CoA reductase mediated diseases such as dyslipidemia and other cardiovascular diseases such as myocardial infarction and stroke.

At the time of priority filing, there were no known polymorphisms in the HMG-CoA reductase gene. On Oct. 7, 1999, in PCT Application WO 99/50454, Lander et al published on a Ile to Val polymorphism at position 638 (see FIG. 1B therein).

In the human HMG CoA reductase gene a single donor splice site is used to excise the intron in the 5′ untranslated region. There are multiple mRNAs due to alternative start sites, all of which have short untranslated regions of 68 to 100 nucleotides (“Conservation of promoter sequence but not complex intron splicing pattern in human and hamster genes for 3-hydroxy-3-methylglutaryl coenzyme A reductase”; Mol. Cell. Biol. 7:1881-1893(1987).)

The HMG-CoA reductase gene has been cloned as cDNA and published as EMBL Accession number M11058 (2904 bp) as defined by SEQ ID NO 44. All positions herein of polymorphisms in the coding sequence relate to the position in SEQ ID NO 44 unless stated otherwise or apparent from the context. The protein sequence of the HMG-CoA reductase has also been been published in Luskey K. L. et al “Human 3-hydroxy-3-methylglutaryl coenzyme A reductase. Conserved domains responsible for catalytic activity and sterol-regulated degradation”; J. Biol. Chem. 260:10271-10277(1985).

A partial genomic sequence of HMG-CoA reductase, including the promoter and exon-1, is published as EMBL Accession number M15959 (1227 bp) as defined by SEQ ID NO 45 herein. All positions herein of polymorphisms in the promoter region relate to the position in SEQ ID NO 45 unless stated otherwise or apparent from the context.

All positions herein of polymorphisms in the intron regions relate to the position of the relevant intron sequence disclosed herein unless stated otherwise or apparent from the context.

HMG-CoA reductase is the rate-limiting enzyme for cholesterol synthesis and is regulated via a negative feedback mechanism mediated by sterols and non-sterol metabolites derived from mevalonate, the product of the reaction catalyzed by reductase. Normally in mammalian cells, this enzyme is suppressed by cholesterol derived from the internalization and degradation of LDL via the LDL receptor. Competitive inhibitors (termed “statins”) of the reductase induce the expression of LDL receptors in the liver, which in turn increases the catabolism of plasma LDL and lowers the plasma concentration of cholesterol, an important determinant of atherosclerosis.

The sequence coding for the highly conserved membrane bound region of the protein is located at positions 51-1067, that coding for the linker part of the protein at positions 1068-1397 and for the strongly conserved water-soluble catalytic part at positions 1398-2714.

One approach is to use knowledge of polymorphisms to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The present invention is based on the discovery of the genomic structure of HMG-CoA reductase and polymorphism therein. In particular, we have found one single nucleotide polymorphism (SNP) in the coding sequence of the HMG-CoA reductase gene, 2 SNPs in the promoter sequence of the HMG-CoA reductase gene and 5 SNPs in the intron sequence of the HMG-CoA reductase gene as well as the genomic structure of the gene and novel sequence allowing the discovery of SNPs in the exons and introns of the gene.

According to one aspect of the present invention there is provided a method for the diagnosis of a single nucleotide polymorphism in HMG-CoA reductase in a human, which method comprises determining the sequence of the nucleic acid of the human at at least one polymorphic position and determining the status of the human by reference to polymorphism in the HMG-CoA reductase gene. Preferred polymorphic positions are one or more of the following positions:

position 1962 in the coding sequence of the HMG-CoA reductase gene as defined by the position in SEQ ID NO: 44, and/or

positions 46 or 267 in the promoter sequence of the HMG-CoA reductase gene as defined by the positions in SEQ ID NO: 45; and/or

position 129 in intron 2 as defined by the position in SEQ ID NO:20,

position 550 in intron 5 as defined by the position in SEQ ID NO: 24,

position 37 in intron 15 as defined by the position in SEQ ID NO:37, or

position 345 in intron 18 as defined by the position in SEQ ID NO:40 of the HMG-CoA reductase gene.

According to another aspect of the present invention there is provided a method for the diagnosis of a single nucleotide polymorphism in HMG-CoA reductase in a human, which method comprises determining the sequence of the nucleic acid of the human at at least one polymorphic position and determining the status of the human by reference to polymorphism in the HMG-CoA reductase gene. Preferred polymorphic positions are one or more of the following positions:

position 1962 in the coding sequence of the HMG-CoA reductase gene as defined by the position in SEQ ID NO: 44, and/or

positions 46 or 267 in the promoter sequence of the HMG-CoA reductase gene as defined by the positions in SEQ ID NO: 45; and/or

position 129 in intron 2 as defined by the position in SEQ ID NO:20,

position 550 in intron 5 as defined by the position in SEQ ID NO: 24,

position 558 in intron 14 as defined by the position in SEQ ID NO:36, or position 345 in intron 18 as defined by the position in SEQ ID NO:40 of the HMG-CoA reductase gene.

The term human includes both a human having or suspected of having a HMG-CoA reductase mediated disease and an asymptomatic human who may be tested for predisposition or susceptibility to such disease. At each position the human may be homozygous for an allele or the human may be a heterozygote.

The term single nucleotide polymorphism includes single nucleotide substitution, nucleotide insertion and nucleotide deletion which in the case of insertion and deletion includes insertion or deletion of one or more nucleotides at a position of a gene.

In one embodiment of the invention preferably the method for diagnosis described herein is one in which the single nucleotide polymorphism at position 1962 of the coding sequence is presence of A and/or G.

In one embodiment of the invention preferably the method for diagnosis described herein is one in which the single nucleotide polymorphism at position 46 of the promoter is presence of T and/or C.

In one embodiment of the invention preferably the method for diagnosis described herein is one in which the single nucleotide polymorphism at position 267 of the promoter is presence of C and/or G.

In another embodiment of the invention preferably the method for diagnosis described herein is one in which the single nucleotide polymorphism at position 129 of intron 2 is the presence or absence of an insertion of AA.

In one embodiment of the invention preferably the method for diagnosis described herein is one in which the single nucleotide polymorphism at position 550 of intron 5 is presence of T and/or A.

In one embodiment of the invention preferably the method for diagnosis described herein is one in which the single nucleotide polymorphism at position 37 of intron 15 is presence of A and/or G.

In one embodiment of the invention preferably the method for diagnosis described herein is one in which the single nucleotide polymorphism at position 345 of intron 18 is presence of T and/or C.

The method for diagnosis is preferably one in which the sequence is determined by a method selected from amplification refractory mutation system and restriction fragment length polymorphism.

Allelic variation at position 1962 consists of a single base substitution from A (the published base), preferably to G.

Allelic variation at position 46 consists of a single base substitution from C (the published case), preferably to G.

Allelic variation at position 267 consists of a single base substitution from T (the published base), preferably to C.

Allelic variation at position 129 consists of a presence or absence of insertion, preferably to presence or absence of the insertion of AA.

Allelic variation at position 550 consists of a single base substitution from T, preferably to A.

Allelic variation at position 37 consists of a single base substitution from A, preferably to G.

Allelic variation at position 345 consists of a single base substitution from T, preferably to C.

The status of the individual may be determined by reference to allelic variation at any one, two, three, four, five, six or seven or more positions.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| FRET | Fluorescence resonance energy transfer |
| HDL | high density lipoprotein |
| HMG-CoA | 3-hydroxy-3-methylglutaryl-coenzyme A |
| LCR | Ligase chain reaction |
| LDL | low density lipoprotein |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

TABLE 1
Mutation Detection Techniques

General: DNA sequencing, Sequencing by hybridisation
Scanning: PTT*, SSCP, DGGE, TGGE, Cleavase, Heteroduplex analysis, CMC, Enzymatic
mismatch cleavage
* Note: not useful for detection of promoter polymorphisms.
Hybridisation Based

Solid phase hybridisation: Dot blots, MASDA, Reverse dot blots,

Oligonucleotide arrays (DNA Chips)

Solution phase hybridisation: Taqman ™ - US-5210015 & US-5487972 (Hoffmann-La

Roche), Molecular Beacons - Tyagi et al (1996), Nature Biotechnology, 14, 303; WO
95/13399 (Public Health Inst., New York)
Extension Based: ARMS ™, ALEX ™ - European Patent No. EP 332435 B1 (Zeneca
Limited), COPS - Gibbs et al (1989), Nucleic Acids Research, 17, 2347.
Incorporation Based: Mini-sequencing, APEX
Restriction Enzyme Based: RFLP, Restriction site generating PCR
Ligation Based: OLA
Other: Invader assay

TABLE 2
Signal Generation or Detection Systems

Fluorescence: FRET, Fluorescence quenching, Fluorescence polarisation - United Kingdom
Patent No. 2228998 (Zeneca Limited)
Other: Chemiluminescence, Electrochemiluminescence, Raman, Radioactivity, Colorimetric,
Hybridisation protection assay, Mass spectrometry

TABLE 3
Further Amplification Methods

SSR, NASBA, LCR, SDA, b-DNA

Preferred mutation detection techniques include ARMS™, ALEX™, COPS, Taqman, Molecular Beacons, RFLP, and restriction site based PCR and FRET techniques.

Particularly preferred methods include ARMS™ and RFLP based methods. ARMS™ is an especially preferred method.

In a further aspect, the diagnostic methods of the invention are used to assess the pharmacogenetics of therapeutic compounds in the treatment of HMG-CoA reductase mediated diseases.

Assays, for example reporter-based assays, may be devised to detect whether one or more of the above polymorphisms affect transcription levels and/or message stability.

Individuals who carry particular allelic variants of the HMG-CoA reductase gene may therefore exhibit differences in their ability to regulate protein biosynthesis under different physiological conditions and will display altered abilities to react to different diseases. In addition, differences in protein regulation arising as a result of allelic variation may have a direct effect on the response of an individual to drug therapy. The diagnostic methods of the invention may be useful both to predict the clinical response to such agents and to determine therapeutic dose.

In a further aspect, the diagnostic methods of the invention, are used to assess the predisposition and/or susceptibility of an individual to diseases mediated by HMG-CoA reductase. This may be particularly relevant in the development of hyperlipoproteinemia and cardiovascular disease and the present invention may be used to recognise individuals who are particularly at risk from developing these conditions.

In a further aspect, the diagnostic methods of the invention are used in the development of new drug therapies which selectively target one or more allelic variants of the HMG-CoA reductase gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes.

According to another aspect of the present invention there is provided a human HMG-CoA reductase gene or its complementary strand comprising a polymorphism, preferably corresponding with one or more of positions defined herein or a fragment thereof of at least 20 bases comprising at least one polymorphism.

Fragments are at least 17 bases, more preferably at least 20 bases, more preferably at least 30 bases.

According to another aspect of the present invention there is provided a polynucleotide comprising at least 20 bases of the human HMG-CoA reductase gene and comprising a polymorphism selected from any one of the following:

| Region | SEQ ID | Position | Polymorphism |
|---|---|---|---|
| Exon 15 | SEQ ID NO: 44 | 1962 | A → G |
| promoter | SEQ ID NO: 45 | 46 | C → G |
| promoter | SEQ ID NO: 45 | 267 | T → C |
| Intron 2 | SEQ ID NO: 20 | 129 | CT → CAAT |
| Intron 5 | SEQ ID NO: 24 | 550 | T → A |
| Intron 15 | SEQ ID NO: 37 | 37 | A → G |
| Intron 18 | SEQ ID NO: 40 | 345 | T → C |

In another embodiment the following polymorphisms are preferred:

| Region | SEQ ID | Position | Polymorphism |
|---|---|---|---|
| promoter | SEQ ID NO: 45 | 46 | C → G |
| promoter | SEQ ID NO: 45 | 267 | T → C |
| Intron 2 | SEQ ID NO: 20 | 129 | CT → CAAT |
| Intron 5 | SEQ ID NO: 24 | 550 | T → A |
| Intron 15 | SEQ ID NO: 37 | 37 | A → G |
| Intron 18 | SEQ ID NO: 40 | 345 | T → C |

According to another aspect of the present invention there is provided a human HMG-CoA reductase gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The invention further provides a nucleotide primer which can detect a polymorphism of the invention.

According to another aspect of the present invention there is provided an allele specific primer capable of detecting a HMG-CoA reductase gene polymorphism, preferably at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a HMG-CoA reductase gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a HMG-CoA reductase gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the single nucleotide polymorphisms of this invention may be used as genetic markers in linkage studies. This particularly applies to the polymorphisms of relatively high frequency in introns 5 and 18 (see below). The HMG-CoA reductase gene has been mapped to chromosome 5q13.3-q14 (Luskey K. L., Stevens B.; RT “Human 3-hydroxy-3-methylglutaryl coenzyme A reductase. Conserved domains responsible for catalytic activity and sterol-regulated degradation”; J. Biol. Chem. 260:10271-10277 (1985)). Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWF:Ag levels: identification of three novel single nucleotide polymorphisms in the vWF gene promoter. Blood (1999) 93:4277-83, Keightley A M, Lam Y M, Brady J N, Cameron C L, Lillicrap D).

According to another aspect of the present invention there is provided a polynucleotide sequence comprising any one of the intron sequences of HMG-CoA reductase defined in any one of SEQ ID NOS: 18-41 herein, an allelic variant thereof, a complementary strand thereof or a fragment thereof. A fragment is at least 17 bases, more preferably at least 20 bases, more preferably at least 30 bases. Preferably the allelic variant is one of the SNPs described herein.

According to another aspect of the invention there is provided a polynucleotide sequence comprising any one of the intron sequences of HMG-CoA reductase defined in any one of SEQ ID NOS: 18-41 and 54 or a complementary strand thereof or a sequence at least 90% homologous thereto.

The degree of homology may be any of the following: at least 80%, 85%, 90%, 95%, 96%, 97%, 98%, or 99% homology. Homology is determined as follows. “Homology” is a measure of the identity of nucleotide sequences or amino acid sequences. In order to characterize the homology, subject sequences are aligned so that the highest order homology (match) is obtained. “Identity” per se has an art-recognized meaning and can be calculated using published techniques. Computer program methods to determine identity between two sequences, for example, include DNAStar software (DNAStar Inc., Madison, Wis.); the GCG program package (Devereux, J., et al., Nucleic Acids Research (1984) 12(1):387); BLASTP, BLASTN, FASTA (Atschul, S. F. et al., J Molec Biol (1990) 215:403). Homology (identity) as defined herein is determined conventionally using the well known computer program, BESTFIT (Wisconsin Sequence Analysis Package, Version 8 for Unix, Genetics Computer Group, University Research Park, 575 Science Drive, Madison, Wis. 53711). When using BESTFIT or any other sequence alignment program to determine whether a particular sequence is, for example, about 80% homologous to a reference sequence, according to the present invention, the parameters are set such that the percentage of identity is calculated over the full length of the reference nucleotide sequence or amino acid sequence and that gaps in homology of up to about 20% of the total number of nucleotides in the reference sequence are allowed. Eighty percent of homology is therefore determined, for example, using the BESTFIT program with parameters set such that the percentage of identity is calculated over the fill length of the reference sequence and gaps of up to 20% of the total number of amino acids in the reference sequence are allowed, and wherein up to 20% of the amino acid residues in the reference sequence may be deleted or substituted with another amino acid, or a number of amino acids up to 20% of the total amino acid residues in the reference sequence may be inserted into the reference sequence. When comparing two sequences, the reference sequence is generally the shorter of the two sequences. This means that for example, if a sequence of 50 nucleotides in length with precise complementarity to a 50 nucleotide region within a 100 nucleotide polypeptide is compared there is 100% identity/homology as opposed to only 50% identity/homology. Percent homologies are likewise determined, for example, to identify preferred species, within the scope of the claims appended hereto, which reside within the range of about 80 percent to 100 percent homology.

According to another aspect of the invention there is provided a polynucleotide sequence comprising any one of the intron sequences of HMG-CoA reductase defined in any one of SEQ ID NOS: 18-41 and 54 or a complementary strand thereof or a sequence that hybridises thereto under stringent conditions. As used herein, stringent conditions are those conditions which enable sequences that possess at least 80%, preferably at least 90% and more preferably at least 95% sequence homology to hybridise together. Thus, nucleic acids which can hybridise to the nucleic acid of SEQ ID No. 18-41 or 54, or the complementary strand thereof, include nucleic acids which have at least 80%, preferably at least 90%, more preferably at least 95%, still more preferably at least 98% sequence homology and most preferably 100% homology. An example of a suitable hybridisation solution when a nucleic acid is immobilised on a nylon membrane and the probe nucleic acid is greater than 500 bases or base pairs is: 6×SSC (saline sodium citrate), 0.5% SDS (sodium dodecyl sulphate), 100 mg/ml denatured, sonicated salmon sperm DNA. The hybridisation being performed at 68° C. for at least 1 hour and the filters then washed at 68° C. in 1×SSC, or for higher stringency, 0.1×SSC/0.1% SDS. An example of a suitable hybridisation solution when a nucleic acid is immobilised on a nylon membrane and the probe is an oligonucleotide of between 12 and 50 bases is: 3M trimethylammonium chloride (TMACl), 0.01M sodium phosphate (pH 6.8), 1 mM EDTA (pH 7.6), 0.5% SDS, 100 mg/ml denatured, sonicated salmon sperm DNA and 0.1 dried skimmed milk. The optimal hybridisation temperature (Tm) is usually chosen to be 5° C. below the Ti of the hybrid chain. Ti is the irreversible melting temperature of the hybrid formed between the probe and its target. If there are any mismatches between the probe and the target, the Tm will be lower. As a general guide, the recommended hybridisation temperature for 17-mers in 3M TMACl is 48-50° C.; for 19-mers, it is 55-57° C.; and for 20-mers, it is 58-66° C.

Novel sequence disclosed herein, may be used in another embodiment of the invention to regulate expression of the gene in cells by the use of anti-sense constructs. To enable methods of down-regulating expression of the gene of the present invention in mammalian cells, an example antisense expression construct can be readily constructed for instance using the pREP10 vector (Invitrogen Corporation). Transcripts are expected to inhibit translation of the gene in cells transfected with this type construct. Antisense transcripts are effective for inhibiting translation of the native gene transcript, and capable of inducing the effects (e.g., regulation of tissue physiology) herein described. Oligonucleotides which are complementary to and hybridizable with any portion of novel gene mRNA disclosed herein are contemplated for therapeutic use. Suitable antisense targets include novel intron/exon junctions disclosed herein. U.S. Pat. No. 5,639,595, Identification of Novel Drugs and Reagents, issued Jun. 17, 1997, wherein methods of identifying oligonucleotide sequences that display in vivo activity are thoroughly described, is herein incorporated by reference. Expression vectors containing random oligonucleotide sequences derived from previously known polynucleotides are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material.

Antisense nucleotide molecules can be synthesized for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, Hybrid Oligonucleotide Phosphorothioates, issued Jul. 29, 1997, and U.S. Pat. No. 5,652,356, Inverted Chimeric and Hybrid Oligonucleotides, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis.

According to another aspect of the present invention there is provided a method of treating a human in need of treatment with a HMG-CoA reductase inhibitor drug in which the method comprises:

i) diagnosis of a single nucleotide polymorphism in HMG-CoA reductase gene in the human, which diagnosis preferably comprises determining the sequence of the nucleic acid at one or more of the following positions:

position 1962 in the coding sequence of the HMG-CoA reductase gene as defined by the position in SEQ ID NO: 44, and/or

positions 46 or 267 in the promoter sequence of the HMG-CoA reductase gene as defined by the positions in SEQ ID NO: 45; and/or

position 129 in intron 2 as defined by the position in SEQ ID NO:20,

position 550 in intron 5 as defined by the position in SEQ ID NO: 24,

position 37 in intron 15 as defined by the position in SEQ ID NO:37, or

position 345 in intron 18 as defined by the position in SEQ ID NO:40 of the HMG-CoA reductase gene.

and determining the status of the human by reference to polymorphism in the HMG-CoA reductase gene; and

ii) administering an effective amount of a HMG-CoA reductase inhibitor.

Preferably determination of the status of the human is clinically useful. Examples of clinical usefulness include deciding which antagonist drug or drugs to administer and/or in deciding on the effective amount of the drug or drugs. Statins already approved for use in humans include atorvastatin, cerivastatin, fluvastatin, pravastatin and simvastatin. The reader is referred to the following references for further information on HMG-CoA reductase inhibitors: Drugs and Therapy Perspectives (May 12, 1997), 9: 1-6; Chong (1997) Pharmacotherapy 17:1157-1177; Kellick (1997) Formulary 32: 352; Kathawala (1991) Medicinal Research Reviews, 11: 121-146; Jahng (1995) Drugs of the Future 20: 387-404, and Current Opinion in Lipidology, (1997), 8, 362-368. Another statin drug of note is compound 3a (S-4522) in Watanabe (1997) Bioorganic and Medicinal Chemistry 5: 437-444.

According to another aspect of the present invention there is provided use of a HMG-CoA reductase antagonist drug in preparation of a medicament for treating a HMG-CoA reductase mediated disease in a human diagnosed as having a single nucleotide polymorphism therein, preferably at one or more of the positions defined herein.

According to another aspect of the present invention there is provided a pharmaceutical pack comprising HMG-CoA reductase antagonist drug and instructions for administration of the drug to humans diagnostically tested for a single nucleotide polymorphism therein, preferably at one or more of the positions defined herein.

According to another aspect of the present invention there is provided an allelic variant of human HMG-CoA reductase polypeptide having a valine at position 638 or a fragment thereof comprising at least 10 amino acids provided that the fragment comprises the allelic variant at position 638.

Fragments of polypeptide are at least 10 amino acids, more preferably at least 15 amino acids, more preferably at least 20 amino acids.

According to another aspect of the present invention there is provided an antibody specific for an allelic variant of human HMG-CoA reductase polypeptide having a valine at position 638 or a fragment thereof comprising at least 10 amino acids provided that the fragment comprises the valine at position 638.

Antibodies can be prepared using any suitable method. For example, purified polypeptide may be utilized to prepare specific antibodies. The term “antibodies” is meant to include polycional antibodies, monoclonal antibodies, and the various types of antibody constructs such as for example F(ab′)₂, Fab and single chain Fv. Antibodies are defined to be specifically binding if they bind the 1638V variant of HMG-CoA reductase with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a diagnostic kit comprising an antibody of the invention.

The invention will now be illustrated but not limited by reference to the following Examples. All temperatures are in degrees Celsius.

In the Examples below, unless otherwise stated, the following methodology and materials have been applied.

AMPLITAQ™ available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989).

Electropherograms were obtained in a standard manner: data was collected by ABI377 data collection software and the wave form generated by ABI Prism sequencing analysis (2.1.2).

### EXAMPLE 1

Identification of Polymorphisms

1. Methods

DNA Preparation

DNA was prepared from frozen blood samples collected in EDTA following protocol I (Molecular Cloning: A Laboratory Manual, p392, Sambrook, Fritsch and Maniatis, 2^{nd} Edition, Cold Spring Harbor Press, 1989) with the following modifications. The thawed blood was diluted in an equal volume of standard saline citrate instead of phosphate buffered saline to remove lysed red blood cells. Samples were extracted with phenol, then phenol/chloroform and then chloroform rather than with three phenol extractions. The DNA was dissolved in deionised water.

Template Preparation

Templates were prepared by PCR using the oligonucleotide primers and annealing temperatures set out below. The extension temperature was 72° and denaturation temperature 94°. Generally 50 ng of genomic DNA was used in each reaction and subjected to 35 cycles of PCR. Where described below, the primary fragment was diluted 1/100 and two microlitres were used as template for amplification of secondary fragments. PCR was performed in two stages (primary fragment then secondary fragment) to ensure specific amplification of the desired target sequence.

Single Nucleotide Polymorphism at Position 1962 of SEQ ID NO: 44

This polymorphism was detected by amplification of a primary fragment from genomic DNA, followed by amplification of a secondary fragment, followed by dye primer sequencing with M13F primer:

Primary Fragment

Forward Oligo, SEQ ID NO: 1

Reverse Oligo, SEQ ID NO: 2

Annealing Temp 68°

Time 1 min

Secondary Fragment

Forward Oligo, SEQ ID NO: 3

Reverse Oligo, SEQ ID NO: 4

Annealing Temp 69°

Time 1 min

Single Nucleotide Polymorphisms at Positions 46 and 267 of SEQ ID NO: 45

These polymorphisms were detected by amplification of a primary fragment from genomic DNA, followed by dye terminator sequencing using the same oligos.

Forward Oligo SEQ ID NO: 5

Reverse Oligo SEQ ID NO: 6

Annealing Temp 64°

Time 2 min

Single Nucleotide Polymorphisms at Position 129 of HMG CoA Reductase Intron 2 Sequence (SEQ ID NO: 20)

This polymorphism was detected by amplification of a primary fragment from genomic DNA, followed by dye terminator sequencing.

Primary Fragment

Forward Oligo SEQ ID NO: 7

Reverse Oligo SEQ ID NO: 8

Annealing Temp 53°

Time 1 min

Dye terminator sequencing oligo SEQ ID NO: 9

Single Nucleotide Polymorphisms at Position 550 of HMG CoA Reductase Intron 5 Sequence SEQ ID NO: 24 (T to A).

This polymorphism was detected by amplification of a primary fragment from genomic DNA, followed by dye primer sequencing with M13F primer:

Primary Fragment

Forward Oligo SEQ ID NO: 42

Reverse Oligo SEQ ID NO: 43

Annealing Temp 69°

Time 1 min

Single Nucleotide Polymorphisms at Position 37 of HMG CoA Reductase Intron 15 Sequence SEQ ID NO: 37 (A to G).

This polymorphism was detected by amplification of a primary fragment from genomic DNA, followed by amplification of a secondary fragment, followed by dye primer sequencing with M13F primer:

Primary Fragment

Forward Oligo SEQ ID NO: 10

Reverse Oligo SEQ ID NO: 11

Annealing Temp 68°

Time 1 min

Secondary Fragment

Forward Oligo SEQ ID NO: 12

Reverse Oligo SEQ ID NO: 13

Annealing Temp 69°

Time 1 min

Single Nucleotide Polymorphisms at Position 345 of HMG CoA Reductase Intron 18 Sequence

This polymorphism was detected by amplification of a primary fragment from genomic DNA, followed by dye terminator sequencing.

Primary Fragment

Forward Oligo SEQ ID NO: 14

Reverse Oligo SEQ ID NO: 15

Annealing Temp 58°

Time 1 min

Dye Terminator Sequencing Oligo SEQ ID NO: 16

Dye Primer Sequencing

Dye-primer sequencing using M13 forward and reverse primers was as described in the ABI protocol P/N 402114 for the ABI Prism™ dye primer cycle sequencing core kit with “AmpliTaq FS”% DNA polymerase, modified in that the annealing temperature was 45° and DMSO was added to the cycle sequencing mix to a final concentration of 5%.

The extension reactions for each base were pooled, ethanol/sodium acetate precipitated, washed and resuspended in formamide loading buffer.

4.25% Acrylamide gels were run on an automated sequencer (ABI 377, Applied Biosystems).

Dye Terminator Sequencing

Dye-terminator sequencing was as described in the ABI protocol P/N 4303150 for the ABI Prism™ Big Dye terminator cycle sequencing core kit with “AmpliTaq FS”™ DNA polymerase.

The extension reactions were ethanol/sodium acetate precipitated, washed and resuspended in formamide loading buffer.

4.25% Acrylamide gels were run on an automated sequencer (ABI 377, Applied Biosystems).

2. Results

Exon-Intron Organisation of the Human HMG-CoA Reductase Gene

Exon sequences are in capital letters: intron sequences (where shown) are in lowercase letters. The number shown immediately below the DNA sequence denotes the nucleotide position from SEQ ID NO: 44 at which the intron interrupts the HMG CoA reductase mRNA. The 5′ boundary and sequence of intron 1 are as described by K. L. Luskey, Mol. Cell. Biol. 7:1881-1893 (1987), Medline ref. No.87257890.

| | Sequence of Exon-Intron Junctions | | |
|---|---|---|---|
| Intron no. | 5′ Boundary 3′ Boundary | Intron size (Kb) | |
| 1. | GAT CTG GAG gtgagg(SEQ ID NO: 17)..ATG TTG TCA | 4.5 | approx |
| | 51 | | |
| 2. | TTT GAG GAG .......... GAT GTT TTG | 1.2 | approx |
| | 215 216 | | |
| 3. | ATA TTT TGG .............. GTA TTG CTG | 0.28 | |
| | 327 328 | | |
| 4. | AGG CTT GAA ............ TGA AGC TTT | 1.222 | |
| | 415 416 | | |
| 5. | AAC TCA CAG ............ GAT GAA GTA | 1.7 | approx |
| | 500 501 | | |
| 6. | CCA TGT CAG ..... GGG TAC GTC | 2 | approx |
| | 606 607 | | |
| 7. | GTA TTA GAG ............. CTT TCT CGG | 0.11 | |
| | 713 714 | | |
| 8. | ATG ATT ATG ...... TCT CTA GGC | 0.414 | |
| | 830 831 | | |
| 9. | TCT CTC TAA ........... AAT GAT CAG | 0.12 | |
| | 991 992 | | |
| 10. | AAA GAA AAG ...... TTG AGG TTA | 0.108 | |
| | 1239 1240 | | |
| 11. | AAT GCA GAG ...........AAA GGT GCA | 4 | approx |
| | 1418 1419 | | |
| 12. | TAC TCC TTG ........... GTG ATG GGA | 0.358 | |
| | 1613 1614 | | |
| 13. | GCA ATA GGT ............. CTT GGT GGA | 0.15 | |
| | 1772 1773 | | |
| 14. | CAC TAG CAG .......... ATT TGC ACG | 1.5 | approx |
| | 1930 1931 | | |
| 15. | ATT TCA AAG ...... GGT ACA GAG | 2 | approx |
| | 2036 2037 | | |
| 16. | GTC AGA GAA .... GTA TTA AAG | 0.343 | |
| | 2207 2208 | | |
| 17. | TGT GGA CAG ........ GAT GCA GCA | 0.088 | |
| | 2348 2349 | | |
| 18. | TGT TTG CAG ...... ATG CTA GGT | 0.428 | |
| | 2507 2508 | | |
| 19. | TCA CCA CAG ...... GTC GAA CAT | 0.149 | |
| | 2662 2663 | | |

Polymorphisms

SEQ ID NO: 44

| | | | | | |
|---|---|---|---|---|---|
| Nucleotide 1962 | A/G | Ile/Val (638) | ATA/GTA | ATA | 95.5% |
| | | | | GTA | 4.5% |

The allele frequencies were based on analysis of 22 individuals. A was the published base. This change in amino acid sequence is within the catalytic domain of the polypeptide and may therefore be of particular interest.

SEQ ID NO: 45

| | | | | |
|---|---|---|---|---|
| Nucleotide 46 | C/G | Allele Frequency | C | 95.8% |
| | | | G | 4.2% |
| C was the published base. | | | | |
| Nucleotide 267 | T/C | Allele Frequency | T | 95.8% |
| | | | C | 4.2% |

T was the published base. These changes in the promoter may affect transcript levels. The allele frequencies were based on analysis of 24 individuals.

HMG CoA Reductase Intron 2 Sequence

| Nucleotide 129 of SEQ ID NO: 20 | | Insertion of AA | |
|---|---|---|---|
| Allele Frequency | CT | | 95% |
| | CAAT | | 5% |

Allele frequencies determined in a panel of 20 individuals

HMG CoA Reductase Intron 5 Sequence

| Nucleotide 570 of | | T/A |
|---|---|---|
| SEQ ID NO: 24 | | |
| Allele Frequency | T | 72.7% |
| | A | 27.3% |

The allele frequencies were based on analysis of 22 individuals.

HMG CoA Reductase Intron 15 Sequence

| Nucleotide 37 of | | A/G |
|---|---|---|
| SEQ ID NO: 37 | | |
| Allele Frequency | A | 97.7% |
| | G | 2.3% |

The allele frequencies were based on analysis of 22 individuals.

HMG CoA Reductase Intron 18 Sequence

| Nucleotide 345 of | | |
|---|---|---|
| SEQ ID NO: 40 | | T/C |
| Allele Frequency | C | 61.7% |
| | T | 28.3% |

The allele frequencies were based on analysis of 23 individuals.

Summary of Polymorphisms

| SNP | Ref | Position | Change |
|---|---|---|---|
| Exon 15 | SEQ ID NO: 44 | 1962, 638 | A → G, Ile → Val |
| promoter | SEQ ID NO: 45 | 46 | C → G |
| promoter | SEQ ID NO: 45 | 267 | T → C |
| Intron 2 | SEQ ID NO: 20 | 129 | CT → CAAT |
| Intron 5 | SEQ ID NO: 24 | 550 | T → A |
| Intron 15 | SEQ ID NO: 37 | 37 | A → G |
| Intron 18 | SEQ ID NO: 40 | 345 | T → C |

Intron Sequence

Intron 1 Sequence (Last 634 bp)

SEQ ID NO: 18

Intron 2 Sequence

First 506 bp, SEQ ID NO: 19

Last 230 bp, SEQ ID NO: 20

Intron 3 Sequence (280 bp)

SEQ ID NO: 21

Intron 4 Sequence (1,222 bp)

SEQ ID NO: 22

Intron 5 Sequence (First 850 bp and Last 730 bp)

SEQ ID NO: 23

SEQ ID NO: 24

Intron 6 Sequence (First 492 bp and Last 715 bp)

SEQ ID NO: 25

SEQ ID NO: 26

Intron 7 Sequence (109 bp

SEQ ID NO: 27

Intron 8 Sequence (414 bp)

SEQ ID NO: 28

Intron 9 Sequence (118 bp)

SEQ ID NO: 29

Intron 10 Sequence (108 bp)

SEQ ID NO: 30

Intron 11 Sequence (First 728 bp and Last 291 bp¹) ¹ last 30 bp shown as SEQ ID NO: 32

SEQ ID NO: 31

SEQ ID NO: 54

Intron 12 Sequence (358 bp)

SEQ ID NO: 33

Intron 13 Sequence (150 bp)

SEQ ID NO: 34

Intron 14 Sequence (First 247 bp and Last 594 bp)

SEQ ID NO: 35

SEQ ID NO: 36

Intron 15 Sequence (First 357 bp)

SEQ ID NO: 37

Intron 16 Sequence (342 bp)

SEQ ID NO: 38

Intron 17 Sequence (87 bp)

SEQ ID NO: 39

Intron 18 (427 bp)

SEQ ID NO: 40

Intron 19 Sequence (148 bp)

SEQ ID NO: 41

### EXAMPLE 2

Single Nucleotide Polymorphism at Position 915 of HMG CoA Reductase Intron 4 Sequence SEQ ID No: 22 (Deletion of T)

This polymorphism was detected by amplification of a primary fragment of genomic DNA, followed by a secondary fragment, followed by dye terminator sequencing.

a) Primary Fragment

Forward oligo SEQ ID No: 49, Reverse oligo SEQ ID No: 47

Annealing temperature 55° C., Time 1 min

b) Secondary Fragment

Forward oligo SEQ ID No. 48, Reverse oligo SEQ ID No. 46

Annealing temperature 55° C., Time 1 min

Dye terminator sequencing oligo; SEQ ID No: 50

### Example 3

ARMS™ Diagnostic Assay To Detect Exon 15 Polymorphism

ARMS™ assay technology is described in Chapter 11 of the textbook PCR by C R Newton & A Graham, 2^{nd} Edition, BIOS Scientific Publishers Ltd, Oxford, UK. Below are the primer sequences needed to carry out a diagnostic ARMS™ assay on the exon 15 polymorphism, in order to detect which allele is present.

The following primers amplify a 198 base pair PCR product only if the A allele is present:

Constant primer (forward): SEQ ID NO: 51

A allele specific primer (reverse): SEQ ID NO: 52

Annealing temp. 68° C., Time 45 secs

The following primers amplify a 198 base pair PCR product only if the G allele is present:

Constant primer (forward): SEQ ID NO: 51

G allele specific primer (reverse): SEQ ID NO: 53

Annealing temp. 68° C., Time 45 secs

Sequence Listing Free Text

For SEQ ID NO: 46-49 & 51-53:

<223> Description of Artificial Sequence:PCR primer

For SEQ ID NO: 50:

<223> Description of Artificial Sequence:dye terminator sequencing oligo

1. A method for the diagnosis of a single nucleotide polymorphism in HMG-CoA reductase in a human, which method comprises determining the sequence of the nucleic acid of the human at at least one polymorphic position selected from one or more of the following positions:
position 1962 in the coding sequence of the HMG-CoA reductase gene as defined by the position in SEQ ID NO: 44, and/or
positions 46 or 267 in the promoter sequence of the HMG-CoA reductase gene as defined by the positions in SEQ ID NO: 45; and/or
position 129 in intron 2 as defined by the position in SEQ ID NO:20, and/or
position 550 in intron 5 as defined by the position in SEQ ID NO: 24, and/or
position 37 in intron 15 as defined by the position in SEQ ID NO:37, and/or
position 345 in intron 18 as defined by the position in SEQ ID NO:40 of the HMG-CoA reductase gene, and
determining the status of the human by reference to polymorphism in the HMG-CoA reductase gene.

2. A method according to claim 1 in which the polymorphism is further defined as the following:
the single nucleotide polymorphism at position 1962 of the coding sequence is presence of A and/or G;
the single nucleotide polymorphism at position 46 of the promoter is presence of T and/or C.
the single nucleotide polymorphism at position 267 of the promoter is presence of C and/or G;
the single nucleotide polymorphism at position 129 of intron 2 is the presence or absence of an insertion of AA;
the single nucleotide polymorphism at position 550 of intron 5 is presence of T and/or A;
the single nucleotide polymorphism at position 37 of intron 15 is presence of A and/or G; and
the single nucleotide polymorphism at position 345 of intron 18 is presence of T and/or C.

3. A method according to claim 1 comprising determining the sequence of the nucleic acid of the human at position 1962 in the coding sequence of the HMG-CoA reductase gene as defined by the position in SEQ ID NO: 44 for presence of A and/or G.

4. A method according to claim 2 in which the sequence is determined by a method selected from amplification refractory mutation system and restriction fragment length polymorphism.

5. Use of a method as defined in claim 2 to assess the pharmacogenetics of therapeutic compounds in the treatment of HMG-CoA reductase mediated diseases.

6. An isolated polynucleotide comprising at least 20 bases of the human HMG-CoA reductase gene and comprising a polymorphism selected from any one of the following:

| Region | SEQ ID | Position | Polymorphism |
|---|---|---|---|
| Exon 15 | SEQ ID NO: 44 | 1962 | A → G |
| promoter | SEQ ID NO: 45 | 46 | C → G |
| promoter | SEQ ID NO: 45 | 267 | T → C |
| Intron 2 | SEQ ID NO: 20 | 129 | CT → CAAT |
| Intron 5 | SEQ ID NO: 24 | 550 | T → A |
| Intron 15 | SEQ ID NO: 37 | 37 | A → G |
| Intron 18 | SEQ ID NO: 40 | 345 | T → C |

7. An allele specific primer or an allele specific oligonucleotide probe capable of detecting a HMG-CoA reductase gene polymorphism at one of the positions defined in the table of claim 6.

8. Use of any polymorphism as defined in the table of claim 6 as a genetic marker in linkage studies.

9 A computer readable medium comprising at least one polymorphism as defined in the table of claim 6 stored on the medium.

10 A method of treating a human in need of treatment with a HMG-CoA reductase inhibitor drug in which the method comprises:
i) diagnosis of a single nucleotide polymorphism in HMG-CoA reductase gene in the human, which diagnosis comprises determining the sequence of the nucleic acid at one or more of the following positions:
position 1962 in the coding sequence of the HMG-CoA reductase gene as defined by the position in SEQ ID NO: 44, and/or
positions 46 or 267 in the promoter sequence of the HMG-CoA reductase gene as defined by the positions in SEQ ID NO: 45; and/or
position 129 in intron 2 as defined by the position in SEQ ID NO:20, and/or
position 550 in intron 5 as defined by the position in SEQ ID NO: 24, and/or
position 37 in intron 15 as defined by the position in SEQ ID NO:37, and/or
position 345 in intron 18 as defined by the position in SEQ ID NO:40 of the HMG-CoA reductase gene,
and determining the status of the human by reference to polymorphism in the HMG-CoA reductase gene; and
ii) administering an effective amount of a HMG-CoA reductase inhibitor.

11 An allelic variant of human HMG-CoA reductase polypeptide comprising a valine at position 638 or a fragment thereof comprising at least 10 amino acids provided that the fragment comprises the valine at position 638.

12 A polynucleotide sequence comprising any one of the intron sequences of HMG-CoA reductase defined in any one of SEQ ID NOS: 18-41 and 54 or a complementary strand thereof or a sequence at least 90% homologous thereto.
